SEQUENCE LISTING (14 sequences)

SEQ ID NO: 1 - DNA, 4611 bp, Homo sapiens
gtgtcgctcgctttctgtcagcctctctccctctccctctcccctctccttcctctcgcttcctctctcgcacctgagcgtacgcacctgcccgggcccggctccctcctcctctcccctccctctttccccgcccggccgcgggagcctcgtggctgcgtcaccgccgcccccccagacaagatggacaccgcggaggaagacatatgtagagtgtgtcggtcagaaggaacacctgagaaaccgctttatcatccttgtgtatgtactggcagtattaagtttatccatcaagaatgcttagttcaatggctgaaacacagtcgaaaagaatactgtgaattatgcaagcacagatttgcttttacaccaatttattctccagatatgccttcacggcttccaattcaagacatatttgctggactggttacaagtattggcactgcaatacgatattggtttcattatacacttgtggcctttgcatggttgggagttgttcctcttacagcatgccgcatctacaagtgcttgtttactggctccgtgagctcactactgacgctgccattagatatgctgtcaacggaaaatttgttggcagattgtttgcagggttgttttgtggtgacgtgcacactgtgtgcattcatcagcctggtgtggttgagagagcagatagtccatgggggagcaccaatttggttggagcatgctgccccaccgttcaatgctgcggggcatcaccaaaatgaggctccagcaggaggaaatggtgcagaaaatgttgctgctgatcagcctgctaacccaccagctgagaacgcagtggtgggggaaaaccctgatgcccaggatgaccaggcagaagaggaggaggaggacaatgaggaggaagatgacgctggtgtggaggatgcggcagatgctaataacggagcccaggatgacatgaattggaatgctttagaatgggaccgagctgctgaagagcttacatgggaaagaatgctaggacttgatggatcactagtttttctggaacatgtcttctgggtggtatctttaaatacactgttcattcttgtttttgcattttgcccttaccatattggtcatttctcccttgttggtttgggatttgaagaacacgtccaagcatctcattttgaaggcctaatcacaaccatagttgggtatatacttttagcaataacactgataatttgtcatggcttggcaactcttgtgaaatttcatagatctcgtcgcttactgggagtctgctatattgttgttaaggtctctttgttagtggtggtagaaattggagtattccctctcatttgtggttggtggctggatatctgttccttggaaatgtttgatgctactctgaaagatcgagaactgagctttcagtcggctccaggtactaccatgtttctgcattggctagtgggaatggtatatgtcttctactttgcctccttcattctactactgagagaggtacttcgacctggtgtcctgtggtttctaaggaatttgaatgatccagatttcaatccagtacaggaaatgatccatttgccaatatataggcatctccgaagatttattttgtcagtgattgtctttggctccattgtcctcctgatgctttggcttcctatacgtataattaagagtgtgctgcctaattttcttccatacaatgtcatgctctacagtgatgctccagtgagtgaactgtccctcgagctgcttctgcttcaggttgtcttgccagcattactcgaacagggacacacgaggcagtggctgaaggggctggtgcgagcgtggactgtgaccgccggatacttgctggatcttcattcttatttattgggagaccaggaagaaaatgaaaacagtgcaaatcaacaagttaacaataatcagcatgctcgaaataacaacgctattcctgtggtgggagaaggccttcatgcagcccaccaagccatactccagcagggagggcctgttggctttcagccttaccgccgacctttaaattttccactcaggatatttctgttgattgtcttcatgtgtataacattactgattgccagcctcatctgccttactttaccagtatttgctggccgttggttaatgtcgttttggacggggactgccaaaatccatgagctctacacagctgcttgtggtctctatgtttgctggctaaccataagggctgtgacggtgatggtggcatggatgcctcagggacgcagagtgatcttccagaaggttaaagagtggtctctcatgatcatgaagactttgatagttgcggtgctgttggctggagttgtccctctccttctggggctcctgtttgagctggtcattgtggctcccctgagggttcccttggatcagactcctcttttttatccatggcaggactgggcacttggagtcctgcatgccaaaatcattgcagctataacattgatgggtcctcagtggtggttgaaaactgtaattgaacaggtttacgcaaatggcatccggaacattgaccttcactatattgttcgtaaactggcagctcccgtgatctctgtgctgttgctttccctgtgtgtaccttatgtcatagcttctggtgttgttcctttactaggtgttactgcggaaatgcaaaacttagtccatcggcggatttatccatttttactgatggtcgtggtattgatggcaattttgtccttccaagtccgccagtttaagcgcctttatgaacatattaaaaatgacaagtaccttgtgggtcaacgactcgtgaactacgaacggaaatctggcaaacaaggctcatctccaccacctccacagtcatcccaagaataaagtagttgtctcaacaacttgaccttcccctttacatgtccttttttgtggacttctctctttggagatttttcccagtgatctctcagcgttgtttttaagttaaatgtatttgacttgtgttctcagcattcagagagcagcggtgtaagattctgctgttctccctggatcttctgacattactgctgtctgagatttgtatatgtgtaaatacaagttccttgataccctaaaaccttggattaaacagaatgtgcattgtacatctttaaacaaaatgtatattaatttattaaatctagttgtcactttattttggacctgctgtgatctcgacaggaaacgtgccacagagcagtagtgcgcaggcaagacttttcagtgacgccttgtggaacgcagttcatgatgtcctagcagctctcactaagggaactgtacattctttctttcttggctattcagaccttaccaagaacgttaaaggaaacaagtagaaatcagcagtggagtgtctgtggtaagaaaacatgaactttatgcttcactgttagttgtttgtggaagttattttgtataacaccaaagctgttgtacatttcctactgcctgatttttttcatgtgtctgtgtttgtaatattgtatagtatcttgtgctaggtgaggaaattatttttaattttgataatttaatattcctagtgtgatcagcattgggagttgggtttcagtggggcatgtctatacttagagaaaaaaagtccaaatgaagattttcatgagtcagcccccccgcccgcccccaccccacacccacatcctctcttttccacacacaactatctgtttattttttgtagcagtggccgaaagtcctgcaaggtcataaatctttcagagtgacatcaccaactgtactgcatcttactggatttaggacttctgagatgcttgtgaagtatagatgtggttgtggtcttagattgacagcattagagaagactggttagaacatctggtctcgctggttagtgcctcgttggctgaggactaggtgtgcatttctcctagcttttcatcaggaaatcccaaagtttccaaagctttttgtttacagaataaaacttcaaataaaaccaattcattatttgtccagaaggaagcttggctgagctggccttttaacataggaatgtatttcgttggaaacattctgaaaaatctcagagaactgaacccttacaaactttgttttccctcataaccaaagcttcaggttagaagtttagaaaaatagaatggttgggtacatgatctaaatgtttaatgctaaaggtatatcgtaagggtagtgtttgtttttgaacgataatttagaagttctcatagaaagcgtataacataggtcttcagaaactataaaagaattttcatatagtattaaaatccatagactaaaatctgagaattttttaacatatgcaagtcagccaaacataagctaccaaaataaagagcaatgtgttctggctgttttatacttcaacaattttttccctaagtggtaagcaattactttaaaacatatttttaaaaacatcggtatcgggagctgcggtggctccggccggttgtcctggcacacaaggaggcgaggctatgcgttcgaggccaacctaggcaaaattgg

SEQ ID NO: 2 - protein, 910 aa, Homo sapiens
MDTAEEDICRVCRSEGTPEKPLYHPCVCTGSIKFIHQECLVQWLKHSRKEYCELCKHRFAFTPIYSPDMPSRLPIQDIFAGLVTSIGTAIRYWFHYTLVAFAWLGVVPLTACRIYKCLFTGSVSSLLTLPLDMLSTENLLADCLQGCFVVTCTLCAFISLVWLREQIVHGGAPIWLEHAAPPFNAAGHHQNEAPAGGNGAENVAADQPANPPAENAVVGENPDAQDDQAEEEEEDNEEEDDAGVEDAADANNGAQDDMNWNALEWDRAAEELTWERMLGLDGSLVFLEHVFWVVSLNTLFILVFAFCPYHIGHFSLVGLGFEEHVQASHFEGLITTIVGYILLAITLIICHGLATLVKFHRSRRLLGVCYIVVKVSLLVVVEIGVFPLICGWWLDICSLEMFDATLKDRELSFQSAPGTTMFLHWLVGMVYVFYFASFILLLREVLRPGVLWFLRNLNDPDFNPVQEMIHLPIYRHLRRFILSVIVFGSIVLLMLWLPIRIIKSVLPNFLPYNVMLYSDAPVSELSLELLLLQVVLPALLEQGHTRQWLKGLVRAWTVTAGYLLDLHSYLLGDQEENENSANQQVNNNQHARNNNAIPVVGEGLHAAHQAILQQGGPVGFQPYRRPLNFPLRIFLLIVFMCITLLIASLICLTLPVFAGRWLMSFWTGTAKIHELYTAACGLYVCWLTIRAVTVMVAWMPQGRRVIFQKVKEWSLMIMKTLIVAVLLAGVVPLLLGLLFELVIVAPLRVPLDQTPLFYPWQDWALGVLHAKIIAAITLMGPQWWLKTVIEQVYANGIRNIDLHYIVRKLAAPVISVLLLSLCVPYVIASGVVPLLGVTAEMQNLVHRRIYPFLLMVVVLMAILSFQVRQFKRLYEHIKNDKYLVGQRLVNYERKSGKQGSSPPPPQSSQE

SEQ ID NO: 3 - DNA, 21 bp, Artificial
  siRNA to human DNA
aagaatgcttagttcaatggc

SEQ ID NO: 4 - DNA, 21 bp, Artificial
  siRNA to human DNA
aaggcctaatcacaaccatag

SEQ ID NO: 5 - DNA, 21 bp, Homo sapiens
gaatggtatatgtcttctact

SEQ ID NO: 6 - DNA, 21 bp, Artificial
  siRNA to human DNA
caataatcagcatgctcgaaa

SEQ ID NO: 7 - DNA, 21 bp, Artificial
  siRNA to human DNA
cagctgcttgtggtctctatg

SEQ ID NO: 8 - DNA, 21 bp, Artificial
  siRNA to human DNA
aaggctcatctccaccacctc

SEQ ID NO: 9 - DNA, 21 bp, Artificial
  siRNA to human DNA
aaacactggacagttctacat

SEQ ID NO: 10 - DNA, 25 bp, Artificial
  siRNA to human DNA
cacgcacacgtgcaggagaatgtag

SEQ ID NO: 11 - DNA, 21 bp, Artificial
  siRNA to human DNA
aaactttccaagctagctact

SEQ ID NO: 12 - DNA, 21 bp, Artificial
  siRNA to human DNA
aagctctctacttactgtgag

SEQ ID NO: 13 - DNA, 16 bp, Artificial
  siRNA to human DNA
aagcctcatttgtgga

SEQ ID NO: 14 - DNA, 21 bp, Artificial
  siRNA to human DNA
gattcgctgacttccttggtt

(19) United States
(12) Patent Application Publication
(10) Pub. No.: US 2008/0090775 A1
(43) Pub. Date: Apr. 17, 2008

(54) Antagonist of TEB4 and Methods of Use

(76) Inventors: Chong Huang, Paoli, PA (US); Lillian Shahied-Arruda, Wayne, PA (US); Gregory M. Arndt, Sydney (AU)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/551,939

(22) Filed: Oct. 23, 2006

Related U.S. Application Data
(60) Provisional application No. 60/731,966, filed on Oct. 31, 2005.

(51) Int. Cl. A61K 31/70; A61P 43/00; C07H 21/04

(52) U.S. Cl. 514/44; 536/24.5

(57) ABSTRACT

The invention concerns antagonists of TEB4 which have therapeutic activity in oncology applications and methods for selecting such antagonists

### CROSS-REFERENCE TO RELATED APPLICATIONS

This application claims priority to provisional application Ser. No, 60/731966, filed Oct. 31, 2005. The invention relates to subject matter disclosed in U.S. patent application Ser. No. 10/957,503 filed Oct. 1, 2004 claiming priority to U.S. Ser. No. 60/508,706 filed on Oct. 6, 2003.

### BACKGROUND OF THE INVENTION

1. Field of the Invention

The invention relates to compositions characterized by the ability to modulate the expression, secretion, or activity of the gene or gene product TEB4. Methods of using the compositions are also provided.

2. Description of the Related Art

TEB4 , a multi-transmembrane domain protein, has been identified as a novel ER (endoplasmic reticulum)-resident ubiquitin ligase. Human TEB4 has homologues in many species and has a number of distinct properties. TEB4 contains a conserved RING (really interesting new gene) finger and 14 predicted transmembrane domains (US20050079613; WO2005/033293). The RING finger of TEB4 and its homologues is situated at the N-terminus.

Studies performed on TEB4 predict that the RING finger domain projects into the cytosol and is shown to catalyse ubiquitin ligation in vitro in a reaction that is ubiquitin Lys48-specific and involves UBC7 (ubiquitin-conjugating enzyme 7) (Hassinck, G. et al. 2005. Biochem. J. 388:647-655). Like E3 enzymes, which are involved in ER-associated protein degradation. TEB4 is an ER degradation substrate itself, promoting its own degradation in a RING finger- and proteasome-dependent manner. Receptors whose degradation is dependent on TEB4 have not been identified and surface expression of MHC class 1, Fas, TfR, CD4 and B7.2 molecules was not influenced by overexpression of TEB4 or its RING finger mutant (Hassink et al. 2005. supra).

The chromosomal location of TEB4 is 5p15, which is a locus known to have aberrant amplification and over-expression in breast and lung cancers. TEB4 (CNGH0002) expression was also shown to be up-regulated under hypoxic conditions (WO2004/033293).

The discovery of the TEB4 gene and its upregulation in both breast tumor cells and endothelial cells that have been exposed to hypoxic conditions, has lead to studies focused on understanding the function of this gene and whether it plays a role in promoting tumorigenesis. TEB4 RNA was found to be over-expressed in lung, breast, and colon cancer tissue.

### SUMMARY OF THE INVENTION

This invention relates to compounds, compositions, and methods useful for modulating TEB4 gene expression using small nucleic acid molecules such as short interfering nucleic acid (siRNA) molecules or other small nucleic acid molecules (sNA) capable of modulating TEB4 expression or activity by RNA interference (RNAi). The use of other small nucleic acid molecules to modulate TEB4 , such as short interfering nucleic acid (siNA), short interfering RNA (siRNA), double-stranded RNA (dsRNA), micro-RNA (miRNA), and short hairpin RNA (shRNA) molecules and methods are also contemplated in the method of the invention.

An sNA or siRNA molecule of the invention can be unmodified or chemically-modified. An sNA or siRNA of the instant invention can be chemically synthesized, expressed from a vector or enzymatically synthesized. The use of chemically-modified siRNA improves various properties of native siRNA molecules through increased resistance to nuclease degradation in vivo and/or through improved cellular uptake. The siRNA molecules of the instant invention provide useful reagents and methods for a variety of therapeutic, veterinary, diagnostic, target validation, genomic discovery, genetic engineering, and pharmacogenomic applications.

In one embodiment, the invention features one or more sNA or siRNA molecules and methods that independently or in combination modulate the expression of TEB4 gene encoding proteins, such as proteins comprising MARCH6 protein associated with the maintenance and/or development of proliferative, metastatic, or angiogenic diseases, traits, conditions and disorders.

### BRIEF DESCRIPTION OF THE SEVERAL VIEWS OF THE DRAWING

FIG. 1 is a graphic representation showing the TEB4 gene and locations of the siRNA constructs prepared.

FIG. 2A-B are graphs showing the affect of MDA-MB-453 cell clones incorporating the indicated siRNA on A) cell index and B) cell proliferation relative to a clone transfected with an empty vector.

FIG. 3 shows the relative effect of the indicated siRNA on MDA-MB-453 cell clones' ability to adhere to fibronectin

FIG. 4A-B shows the relative effect of the indicated siRNA on MDA-MB-453 cell clones' A) ability to migrate and B) invasion.

### DETAILED DESCRIPTION OF THE INVENTION

### Abbreviations

Abs antibodies, polyclonal or monoclonal; Ig immunoglobulin; Mab monoclonal antibody

### Definitions

“Activation” and “stimulation” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compositions derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Proliferative activity” encompasses an activity that promotes, that is necessary for, or that is specifically associated with, e.g., normal cell division, as well as cancer, tumors, dysplasia, cell transformation, metastasis, and angiogenesis.

By “RNA” is meant a molecule comprising at least one ribonucleotide residue. By “ribonucleotide” is meant a nucleotide with a hydroxyl group at the 2′position of a-D-ribofuranose moiety. The terms include double-stranded RNA, single-stranded RNA, isolated RNA such as partially purified RNA, essentially pure RNA, synthetic RNA, recombinantly produced RNA, as well as comprising non-standard nucleotides, such as non-naturally occurring nucleotides or chemically synthesized nucleotides or deoxynucleotides.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference “RNAi” or gene silencing in a sequence-specific manner.

“siRNA” as used herein means a double stranded short interfering RNA molecules of no larger than about 23 nucleotides in length. The scientific literature describes siRNA as mediating the sequence specific degradation of a target MRNA.

“TEB4 ”, “TEB4 gene”, “TEB4 gene product”, “CNGH2”, “CNGH0002” “CNGH0002 gene” “CNGH0002 gene product” are used herein interchangeably and refer to the gene curated by the NCBI provisionally as REFSEQ NM_{—}005885 on Sep. 24, 2005 and its encoded polypeptide, NP_{—}005876.2. Other synonyms include MARCH6, RNF176, KIAA0597, and MARCH-VI and “membrane-associated ring finger (C3HC4) 6”.

Here we show that siRNA sequences were constructed and stably transfected into the breast tumor cell line, MDA MB 435S GFP and clones with varying degrees of TEB4 knock-down were identified. In vitro analysis of these clones has revealed that knock-down of the TEB4 mRNA alters tumor cell proliferation, adhesion, migration and invasion. These findings indicated that TEB4 can play a role in tumorigenesis and is therefore a tumor biomarker and suitable therapeutic target.

### Role of TEB4 in Solid Tumors

The chromosomal location of TEB4 is 5p15, which is a locus known to have aberrant amplification and over-expression in breast and lung cancers. TEB4 (CNGH0002) expression was also shown to be up-regulated under hypoxic conditions (WO2005/033293). Hypoxia occurs in neoplastic tissue as tumor cell proliferation outpaces the process of angiogenesis or neovascularization.

Regulation of oxygen homeostasis is central to all multi-cellular organisms. Almost all mammalian cells express components of a hypoxia response pathway and members of this pathway are conserved in lower organisms such as flies and worms. The hypoxia-inducible transcription factor (HIF), central to this pathway, was identified as a regulator of the genes for glucose transport and glycolytic enzymes, as well as cell differentiation and proliferation factors including EPO (erythropoietin) gene among others such as transferrin, IGF-1R, VEGF, and VEGF receptor Flt-1 (see Semanza, G L. 1999. Ann Rev Cell Dev Biol 15: 551-78 for a review). Tumors are characterized by aerobic glycolysis as noted the by Warburg as early as 1956. Thus, the functional role of TEB4 as a ubiquitin-ligase in tumor progression can now be attributed to its involvement with modulation of cellular proteins of the tumor cell phenotype.

HIF-1 is a heterodimer composed of two members of the basic-Helix-Loop-Helix (bHLH)-containing PER-ARNT-SIM (PAS) domain family; HIF-1α (or the closely related HIF-2α/EPAS-1 or HIF-3α factors) and HIF-1β, also known as the aryl hydrocarbon receptor nuclear translocator (ARNT). Under normoxic conditions HIF-1α is constitutively expressed. However, this subunit is known to be rapidly targeted for proteosome-mediated degradation via a protein-ubiquitin ligase complex containing the product of the von Hippel Lindau tumor suppressor protein (pVHL). pVHL recognizes the oxygen degradation domain (ODD) of HIF-1α only under normoxic conditions. Following exposure to a hypoxic environment, this degradation pathway is blocked, allowing HIF-1α accumulation and subsequent movement to the nucleus where it activates hypoxia-responsive genes.

The finding that TEB4 is a hypoxia responsive gene provided the impetus for further study of TEB4 gene expression in tumor cell proliferation and functional measures of metastatic mechanisms such as migration, invasion, and adhesion.

Proteasome Inhibitor Activity

The proteasome mediates degradation not only of cytosolic and nuclear proteins but also of proteins that reside in the endoplasmic reticulum (ER). As the ER proteins function to move secreted proteins to and from the cell surface and these include receptors essential for cell-recognition, e.g. MHC proteins; nutrient transport, e.g. Glut1 protein; hormone receptors, e.g. EPO receptors, VEGFR; and cytokine and chemokine receptors, e.g. TNFR1 and CCR2; the rate of degradation of these proteins can be seen as essential to the maintenance of the cell surface configuration and display of receptors. Covalent attachment of ubiquitin chains to lysine residues is the main mode of targeting proteins to proteasomes. The attachment of multiple ubiquitin molecules to proteins involves the action of three enzymes, the ubiquitin-activating enzyme, designated E1, a ubiquitin-conjugating/carrier enzyme or E2, and a ubiquitin ligase or E3. The ubiquitin-proteasome pathway plays an essential role in regulating the intracellular concentration of specific proteins, thereby maintaining homeostasis within cells. Inhibition of the 26S proteasome prevents this targeted proteolysis, which can affect multiple signaling cascades within the cell. This disruption of normal homeostatic mechanisms can lead to cell death. Therapeutic agents acting through proteasome pathway have been developed. VELCADE® (bortezomib) is an antineoplastic agent which is a proteasome inhibitor approved to treat multiple myeloma. Bortezomib is a reversible inhibitor of the chymotrypsin-like activity of the 26S proteasome in mammalian cells.

Based on the discoveries of the present invention combined with what is known in the art, the use of TEB4 antagonists can be used alone or in combination with other ubiquitin-proteasome pathway inhibitors to prevent or treat pathologic conditions.

### Agents Useful for TEB4 Antagonism

Gene expression can be modulated in several different ways, including by the use of siRNAs, shRNAs, antisense molecules and DNAzymes. SiRNAs and shRNAs both work via the RNAi pathway and have been successfully used to suppress the expression of genes. RNAi was first discovered in worms and the phenomenon of gene silencing related to dsRNA was first reported in plants by Fire and Mello (Fire et al., 1998. Nature 391: 806) and is thought to be a way for plant cells to combat infection with RNA viruses. In this pathway, the long dsRNA viral product is processed into smaller fragments of 21-25 bp in length by a DICER-like enzyme and then the double-stranded molecule is unwound and loaded into the RNA induced silencing complex (RISC). A similar pathway has been identified in mammalian cells with the notable difference that the dsRNA molecules must be smaller than 30 bp in length in order to avoid the induction of the so-called interferon response, which is not gene specific and leads to the global shut down of protein synthesis in the cell.

Synthetic siRNAs can be designed to specifically target one gene and they can easily be delivered to cells in vitro or in vivo. ShRNAs are the DNA equivalents of siRNA molecules and have the advantage of being incorporated into the cells' genome and then being replicated during every mitotic cycle.

DNAzymes have also been used to modulate gene expression. DNAzymes are catalytic DNA molecules that cleave single-stranded RNA. They are highly selective for the target RNA sequence and as such can be used to down-regulate specific genes through targeting of the messenger RNA.

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13:139-141; and Strauss, 1999, Science, 286, 886). The presence of dsRNA in cells triggers the RNAi response through a mechanism that has yet to be fully characterized. This mechanism appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of a ribonuclease III enzyme referred to as dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21-and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

siRNAs are double stranded RNAs that include the target sequence and its complement. Two uridine residues are added to the 3′ end of the RNAs (Elbashir et al. 2001 Nature 411:494-498).

### Expression of siRNA in Vivo Elicits Gene Silencing

RNA interference (RNAi) is now being used routinely in mammalian cells to study the functional consequences of reducing the expression of specific genes. RNAi is induced by transfecting small interfering RNAs (siRNAs), comprising double-stranded RNA molecules ˜21 nt in length with 2 nt 3′ overhangs (Elbashir et al. 2001 supra), or hairpin-forming 45-50 mer (shRNA) molecules (Paddison, P J, et al., 2002. Genes & Development 16:948-958), that are complementary to the gene of interest. When transfected into mammalian cells, siRNA expression plasmids and have been shown to reduce the levels of both exogenous and endogenous gene products. Although they require more effort to prepare than chemically synthesized or in vitro transcribed siRNAs, the siRNA vectors can provide longer term reduction in target gene expression when coexpressed with a selectable marker (Brummelkamp, T R, et al., 2002. Science 296:550-553).

### Methods of using TEB4 Antagonists

The invention includes methods for preparing pharmaceutical compositions for modulating the transcription, expression, or activity of a TEB4 polypeptide or nucleic acid.

Such methods comprise formulating a pharmaceutically acceptable carrier with an agent that modulates expression or activity of a TEB4 polypeptide or nucleic acid. Such compositions can further include additional active agents. Thus, the invention further includes methods for preparing a pharmaceutical composition by formulating a pharmaceutically acceptable carrier with an agent that modulates expression or activity of a TEB4 polypeptide or nucleic acid and one or more additional active compounds.

### Methods of Treatment

The present invention provides for both prophylactic and therapeutic methods of treating a subject at risk of (or susceptible to) a disorder or having a disorder associated with aberrant expression or activity of a TEB4 polypeptide or nucleic acid and/or in which the a TEB4 polypeptide or nucleic acid is involved.

The present invention provides a method for modulating or treating at least one TEB4 polypeptide or nucleic acid related disease or condition, in a cell, tissue, organ, animal, or patient, as known in the art or as described herein, using at least one TEB4 polypeptide or nucleic acid Antagonist.

Compositions of a TEB4 polypeptide or nucleic acid antagonist may find therapeutic use in the treatment of proliferative, metastatic, or angiogenic diseases, traits, conditions and disorders

These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a TEB4 gene or polypeptide. In one embodiment, the method involves administering an agent (e.g., an agent identified by a screening assay described herein), or combination of agents that modulate (e.g., up-regulates or down-regulates) expression or activity. Inhibition of activity is desirable in situations in which activity or expression is abnormally high or up-regulated and/or in which decreased activity is likely to have a beneficial effect.

The present invention also provides a method for modulating or treating at least one malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), acute promyelocytic leukemia (APL), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, hemangioma, metastatic disease, cancer related bone resorption, cancer related bone pain, and the like.

Disorders characterized by aberrant expression or activity of a TEB4 polypeptide or nucleic acid are further described elsewhere in this disclosure.

In one aspect, the invention provides a method for at least substantially preventing in a subject, a disease or condition associated with an aberrant expression or activity of a TEB4 polypeptide or nucleic acid, by administering to the subject an agent that modulates expression or at least one activity of the gene and, therefore, the polypeptide. Subjects at risk for a disease that is caused or contributed to by aberrant expression or activity of a TEB4 polypeptide or nucleic acid can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

### Methods of Administration

The TEB4 polypeptide or nucleic acid antagonist molecules can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Either the naked nucleic acid antagonist molecules or the engineered vectors comprising the antagonist sequences of the invention can be encapsulated for administration to a subject. In one embodiment, the encapsulated form may be a microparticle, that is comprised of a wall forming material. In another embodiment, the encapsulated form is a lipid vesicle, e.g. a liposome.

The term “microparticle” is synonymous with and includes the terms “microsphere” and “microcapsule”. Preferably the microparticle composition is substantially dry or powder-like, i.e. liquid has not been added to the composition. Some minor amounts of liquid may however remain with the microparticles. The polymeric matrix material of the microparticles present invention can be composed of a biocompatible and biodegradable polymeric material. The term “biocompatible material” is defined as a polymeric material which is not toxic to an animal and not carcinogenic. The matrix material is preferably biodegradable in the sense that the polymeric material should degrade by bodily processes in vivo to products readily disposable by the body and should not accumulate in the body.

The microparticles of the present invention usually have a spherical shape, although irregularly-shaped microparticles are possible. The microparticles vary in size, ranging in diameter from 0.1 microns to 250 microns, more preferably, from 10 or 20 microns to 75 microns and most preferably from 30 microns to 70 microns.

The term “sustained-release” as used herein encompasses the term “controlled-release” and means that the biologically active agent is released from the microparticle polymeric matrix over an extended period of time so as to give continuing or delayed dosage of the treated organism. The controlled-release period can be from a few hours to 1 to 500 days or longer and preferably is from 3 to 60 days.

Suitable wall-forming materials for use in microcapsules include, but are not limited to, poly(dienes) such as poly(butadiene) and the like; poly(alkenes) such as polyethylene, polypropylene, and the like; poly(acrylics) such as poly(acrylic acid) and the like; poly(methacrylics) such as poly(methyl methacrylate), poly(hydroxyethyl methacrylate), and the like; poly(vinyl ethers); poly(vinyl alcohols); poly(vinyl ketones); poly(vinyl halides) such as poly(vinyl chloride) and the like; poly(vinyl nitrites), poly(vinyl esters) such as poly(vinyl acetate) and the like; poly(vinyl pyridines) such as poly(2-vinyl pyridine), poly(5-methyl-2-vinyl pyridine) and the like; poly(styrenes); poly(carbonates); poly(esters); poly(orthoesters); poly(esteramides); poly(anhydrides); poly(urethanes); poly(amides); cellulose ethers such as methyl cellulose, hydroxyethyl cellulose, hydroxypropyl methyl cellulose, and the like; cellulose esters such as cellulose acetate, cellulose acetate phthalate, cellulose acetate butyrate, and the like; poly(saccharides), proteins, gelatin, starch, gums, resins, and the like. See for example International Patent Application No. PCT/GB00/00349 which is incorporated herein by reference. The polymeric materials may be cross-linked.

These materials may be used alone, as physical mixtures blends) or as copolymers (which may be block copolymers). A preferred group of wall-forming materials includes biodegradable polymers such as poly(lactide), poly(glycolide), poly(caprolactone), poly(hydroxybutyrate), and copolymers thereof including but not limited to poly(lactide-co-glycolide), poly(lactide-co-caprolactone) and the like. Again, these polymers may be cross-linked. The copolymers may be block, random or regular copolymers.

The duration of release of the active agent from the microparticle can be adjusted from less than a week to several months or longer by manipulation of various parameters. The amount (level) of biologically active agent released can also be controlled. The parameters include the polymer composition of the controlled-release material, the polymer molecular weight, the polymer:bioactive agent ratio, microparticle diameter and the presence/absence of a release rate modifier in the composition. Other parameters include bound/unbound drug (with respect to a polymer matrix), hydrophobicity of the drug and/or polymer composition and porosity of the polymer matrix.

“Lipid vesicles” refers to any stable micelle or liposome composition comprising vesicle-forming amphipathic lipids including one or two hydrophobic acyl hydrocarbon chains attached to a polar head group and may contain a chemically reactive group, such as an amine, acid, ester, aldehyde or alcohol, at its polar head group. “Pre-formed liposomes” refers to intact, previously formed unilamellar vesicles (SUVs), large unilamellar vesicles (LUVs) or multi-lamellar vesicles (MLVs) lipid vesicles.

Liposomes as well as other micellar lipid vesicles are included in the methods of the invention for incorporation of the TEB4 nucleic acid antagonist in order to act as drug delivery vehicles. The methods of preparation and drug loading procedures for liposomes and the others are well-known in the art. Liposomes can store both nonpolar and polar compounds via interactions with the biocompatible and biodegradable lipid bilayer, or within the aqueous core, respectively.

Lipids suitable for use in the composition of the present invention include those vesicle-forming lipids. Such a vesicle-forming lipid is one which (a) can form spontaneously into unilamellar or bilayer vesicles in water, as exemplified by the diglycerides and phospholipids, or (b) is stably incorporated into lipid structures including unilammellar, bilayered, or rafts.

The vesicle-forming lipids of this type typically have two hydrocarbon chains, usually acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods. Other suitable lipids include glycolipids, cerebrosides and sterols, such as cholesterol.

Cationic lipids are also suitable for use in the liposomes of the invention, where the cationic lipid can be included as a minor component of the lipid composition or as a major or sole component. Such cationic lipids typically have a lipophilic ligand, such as a sterol, an acyl or diacyl chain, and where the lipid has an overall net positive charge. Typically, the head group of the lipid carries the positive charge. Exemplary cationic lipids include 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxyethylammonium bromide (DORIE); N-[1-(2,3-dioleyloxy) propyl]-N,N,N-trimethylammonium chloride (DOTMA); 3 [N-(N′,N′-dimethylaminoethane)carbamoly]cholesterol (DC-Chol); and dimethyldioctadecylammonium (DDAB).

The cationic vesicle-forming lipid may also be a neutral lipid, such as dioleoylphosphatidyl ethanolamine (DOPE) or an amphipathic lipid, such as a phospholipid, derivatized with a cationic lipid, such as polylysine or other polyamine lipids. For example, the neutral lipid (DOPE) can be derivatized with polylysine to form a cationic lipid.

In another embodiment, the vesicle-forming lipid is selected to achieve a specified degree of fluidity or rigidity, to control the stability of the liposome in serum, to control the conditions effective for insertion of the targeting conjugate, as will be described, and to control the rate of release of the entrapped agent in the liposome.

Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures.

On the other hand, lipid fluidity is achieved by incorporation of a relatively fluid lipid, typically one having a lipid phase with a relatively low liquid to liquid-crystalline phase transition temperature, e.g., at or below room temperature.

In an embodiment of the invention, the pre-formed liposomes also include a vesicle-forming lipid derivatized with a hydrophilic polymer. As has been described, for example in U.S. Pat. No. 5,013,556, including such a derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating by presentation of a non-immunogenic outer surface. Such liposomes are also structurally stabilized and are known as sterically-stabilized liposomes

Vesicle-forming lipids suitable for derivatization with a hydrophilic polymer include any of those lipids listed above, and, in particular phospholipids, such as distearoyl phosphatidylethanolamine (DSPE).

Hydrophilic polymers suitable for derivatization with a vesicle-forming lipid include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers.

An exemplary hydrophilic polymer chain is polyethyleneglycol (PEG) having a molecular weight between 500-10,000 daltons, more typically between 1,000-5,000 daltons. Methoxy or ethoxy-capped analogues of PEG are also useful hydrophilic polymers, commercially available in a variety of polymer sizes, e.g., 120-20,000 daltons.

Preparation of vesicle-forming lipids derivatized with hydrophilic polymers has been described, for example in U.S. Pat. No. 5,395,619. Preparation of liposomes including such derivatized lipids has also been described, where typically, between 1-20 mole percent of such a derivatized lipid is included in the liposome formulation.

In another embodiment, the liposomes are composed of distearoylphosphatidylcholine (DSPC): cholesterol (52:45 molar ratio), and contain additionally 3 mol % PEG(2000)-DSPE compared to total lipid. The liposomes are prepared by freeze-thaw cycles and extrusion as described (Huwyler, et al. (1996) Proc Natl Acad Sci USA 93: 14164-14169). Essentially, lipids are first dissolved in chloroform or chloroform/methanol 2:1 vol/vol. A lipid film is prepared by vacuum evaporation using a Rotavapor (Büchi, Switzerland). Dried lipid films are hydrated at 40° C. in 0.01 M PBS or 65o in 0.3 M citrate (pH4.0), such that a final lipid concentration of 10 mM is achieved. Lipids are subjected to five freeze-thaw cycles, followed by extrusion (5 times) at 20° C. through a 100 nm pore-size polycarbonate membrane employing an extruder (Avanti Polar Lipids, Alabaster, Ala.). Extrusion is repeated 9 times using a 50 nm polycarbonate membrane. This procedure produces PEG-derived liposomes with mean vesicle diameters of 150 nm. As has been previously demonstrated (Schnyder, et al. (2004) Biochem J 377:61-67), biotinylated loaded liposomes may be prepared by substituting a portion of the PEG-DSPE with linker lipid (biotin-PEG-DSPE) and dye or drug may encapsulated by adding the active at the hydration step.

Stable nucleic acid-lipid particles (SNALP) useful for encapsulating one or more siRNA molecules, methods of making SNALPs comprising siRNA, SNALPs comprising siRNA and methods of delivering and/or administering the SNALPs to a subject to silence expression of a target gene sequence are taught in US20050064595.

While having described the invention in general terms, the embodiments of the invention will be further disclosed in the following examples.

### EXAMPLE 1

### TEB4 Antagonism Using

TEB4 , a multi-transmembrane domain protein, has been identified as one of the genes that are consistently upregulated in the center region of malignant melanoma tumors. Concordantly, in a microarray analysis of human breast cancer cells, we have identified that TEB4 is upregulated under hypoxic conditions. In an effort to further elucidate the role of TEB4 in cancer, stable MDA MB-435S GFP tumor cell lines expressing shRNAs specific for this gene were developed. The pSilencer 1.0-U6 Vector (sold by Ambion, http://www.ambion.com) was used to facilitate plasmid-based siRNA experiments. pSilencer 1.0-U6 contains a U6 Pol III promoter and sequence elements for cloning and bacterial replication. This vector was developed by Sui and colleagues at Harvard Medical School and has been successfully used to knock down expression of cdk-2 and lamin A/C in HeLa, H1299, U-2 OS and C-33A (cdk-2 only) cells (2002. Proc Natl Acad Sci USA 99(8): 5515-5520). To use the pSilencer 1.0-U6 Vector, the vector is linearized with Apa I and EcoR I. The double-stranded DNA˜55 bp insert sequence should include 4 nucleotide overhangs complementary to the Apa I and EcoR I restriction sites, as well as the sense and antisense sequences of the desired siRNA separated by a small loop sequence. This double-stranded DNA insert is then ligated into the linearized vector and introduced into E. coli cells. The resulting plasmid is produced in E. coli, purified and then transfected into mammalian cells.

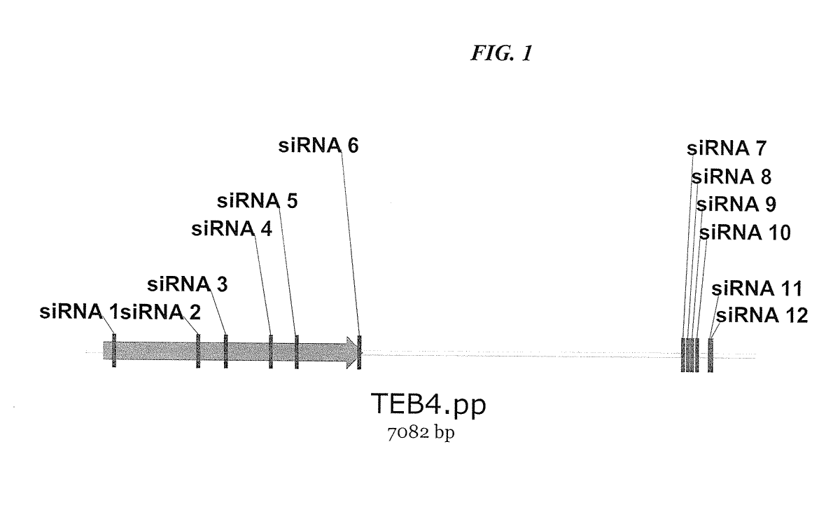

FIG. 1 shows the design of the multiple siRNAs targeting TEB4 provided in Table 1. Delineated in this schematic diagram are the regions of the TEB4 gene that were targeted by siRNA sequences. SiRNA Sequences 2, 6, 8, 9, 10 & 11 proved to be the most effective at knocking down TEB4 RNA expression as determined by QZyme analysis. These same sequences were then cloned into shRNA vectors (pSilencer) and stably transfected into the MDA MB 435S GFP breast tumor cell line.

Four shRNA clones, having varying degrees of TEB4 RNA knock-down ranging from 20-85%, were selected to study in in vitro assays. Experiments focused on determining if reduced TEB4 gene expression could attenuate typical tumor promoting properties such as proliferation rate, adhesion and invasion.

Using an RT-CES cell sensor system (ACEA Biosciences, Inc), the proliferation rates of the knock-down clones were measured in comparison to the parental and vector-control clones. The results showed that the clones with the greatest degree of TEB4 mRNA knock-down also had a decreased proliferation rate, up to 66% in one case.

TABLE 1

| | | |
|---|---|---|
| SEQ ID | SiRNA | |
| NO: | Construct | Sequence |

| | | |
|---|---|---|
| 3 | siRNA #1 | AAG AAT GCT TAG TTC AAT GGC |
| 4 | siRNA #2 | AAG GCC TAA TCA CAA CCA TAG |
| 5 | siRNA #3 | GAA TGG TAT ATG TCT TCT ACT |
| 6 | siRNA #4 | CAA TAA TCA GCA TGC TCG AAA |
| 7 | siRNA #5 | CAG CTG CTT GTG GTC TCT ATG |
| 8 | siRNA #6 | AAG GCT CAT CTC CAC CAC CTC |
| 9 | siRNA #7 | AAA CAC TGG ACA GTT CTA CAT |
| 10 | siRNA #8 | CAC GCA CAC GTG CAG GAG AAT GTAG |
| 11 | siRNA #9 | AAA CTT TCC AAG CTA GCT ACT |
| 12 | siRNA #10 | AAG CTC TCT ACT TAC TGT GAG |
| 13 | siRNA #11 | AAG CCT CAT TTG TGG A |
| 14 | siRNA #12 | GAT TCG CTG ACT TCC TTG GTT |

Knock-down of TEB4 mRNA Expression using shRNA. QZyme analysis of the stably transfected MDA MB 435S GFP cells was perform to measure the levels of mRNA expression relative to the pSilencer vector control. Results showed that there were several clones that exhibited TEB4 mRNA knock-down ranging from 20-85% (Table 2). Clones 8#1, 8#4, 2#5 and 2#6 were selected for further investigation, providing a panel of clones with low, medium and high levels of TEB4 mRNA inhibition to evaluate.

| | Activity | |
|---|---|---|
| Clone | (Percent) | error |
| pSilencer | 100 | 15.4 |
| shRNA-8#1 | 80 | 14.3 |
| shRNA-9#1 | 77 | 12.3 |
| shRNA-2#2 | 75 | 16.5 |
| shRNA-9#3 | 63 | 13.2 |
| shRNA-10#1 | 61 | 9.3 |
| shRNA-8#4 | 48 | 11.4 |
| shRNA-11#4 | 43 | 9.3 |
| shRNA-6#6 | 41 | 9.9 |
| shRNA-2#1 | 40 | 8.5 |
| shRNA-10#3 | 40 | 7.9 |
| shRNA-8#3 | 37 | 6.6 |
| shRNA-11#3 | 37 | 6.6 |
| shRNA-2#4 | 36 | 7.3 |
| shRNA-9#2 | 32 | 6.3 |
| shRNA-11#1 | 32 | 7.6 |
| shRNA-2#3 | 31 | 8.1 |
| shRNA-2#5 | 17 | 4.7 |
| shRNA-2#6 | 17 | 3.3 |
| shRNA-2#7 | 15 | 3.0 |

### EXAMPLE 2

### TEB4 Antagonism Reduces Tumor Cell Proliferation

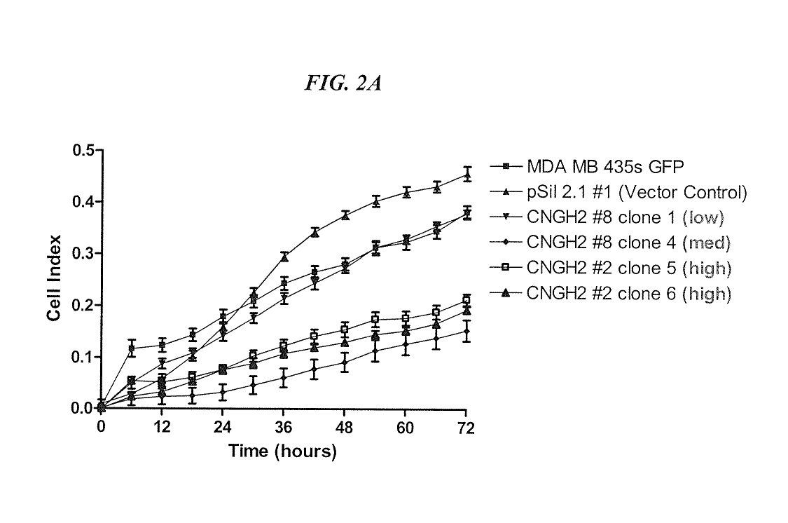

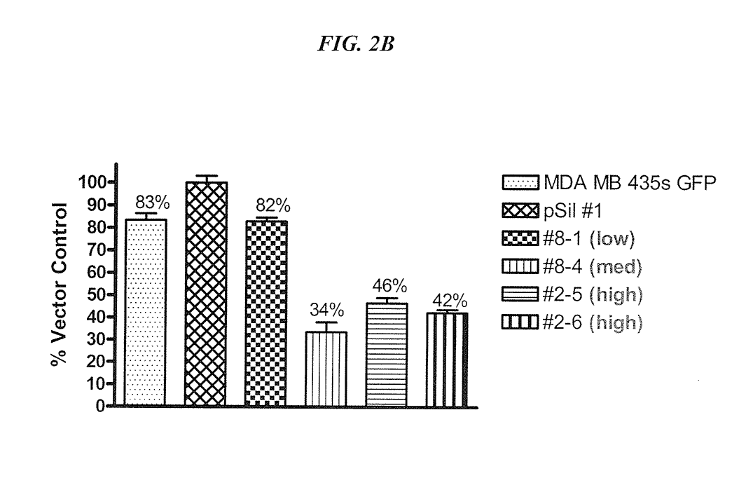

RT-CES Proliferation Assay. 5,000 cells/well of each stable cell line were plated in Complete DMEM Media+200 mg/ml Hygromycin (except parental cells). Readings were taken every 10 minutes for 72 hours on the RT-CES System (ACEA Biosciences) at 37° C. The graph (FIG. 2A) shows each time-point over the 72 hour incubation. The levels of TEB4 knock-down are indicated in the figure legend. FIG. 2B is a bar graph representing the percent vector control for a single time-point (72 hours). The results indicated that clones #8-4, #2-5 and #2-6 exhibited significantly slower growth than the vector control clone.

### EXAMPLE 3

### TEB4 Antagonism Affects Cell Adhesion

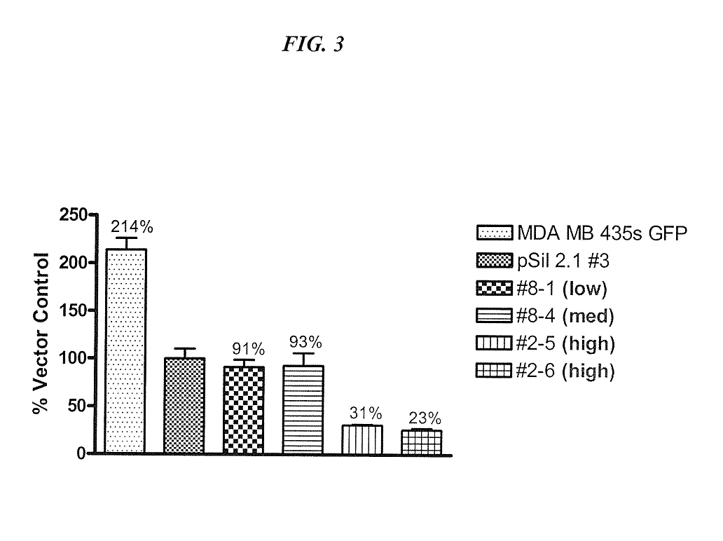

Cell Adhesion Assay: 3×10⁶ cells from each stable cell line were labeled with Calcein AM from the Vybrant Cell Adhesion Kit (Molecular Probes). Cells were washed and 20,000 cells/well in DMEM+0.1% BSA were added to a 96-well plate (Linbro) which had been coated with 10 mg/ml vitronectin. Cells were incubated at 37° C. for 1 hour and the fluorescence was measured on a TECAN plate reader. The percent adhesion relative to the vector control clone is shown in FIG. 3. The levels of TEB4 knock-down are indicated in the figure legend. The results (FIG. 3) showed that clones #2-5 and #2-6 exhibited significantly less adhesion to vitronectin than the vector control cells.

### EXAMPLE 3

### TEB4 Antagonism Affects Cell Migration

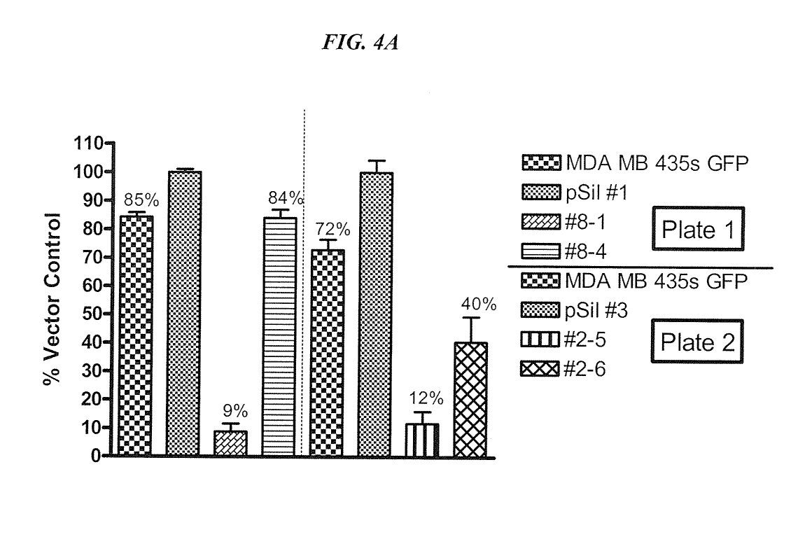

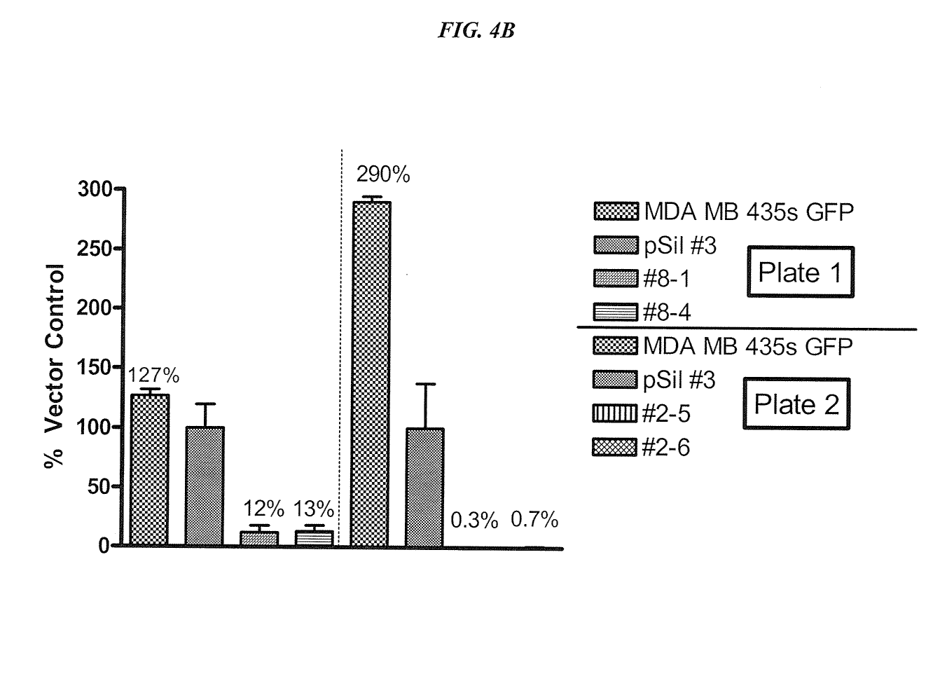

Migration Assay. 1×10⁶ cells (500 ml) of each stable clone were added to the upper chambers of migration wells (Becton Dickinson—24-well format) in DMEM+0.1% BSA. 750 ml of chemoattractant (DMEM+10% FBS) were added to the lower chamber. Cells were incubated for 48 hours, fixed in 2% paraformaldehyde and stained with crystal violet. Cells were quantified using the Phase 3 Imaging System. The results (FIG. 4A.) showed that although all the TEB4 knock-down clones migrated slower than the vector control clone, clones #8-1, #2-5 and #2-6 were significantly slower.

Matrigel Invasion Assay. Followed the same protocol as Migration assay except Invasion Transwell plates (Becton Dickinson-24 well format) which were precoated with matrigel were used. Again, all TEB4 knock-down clones proved to be significantly less invasive than the vector control cells (FIG. 4B).

What is claimed:

1. An isolated short interfering nucleic acid (siNA) molecule that directs cleavage of TEB4 RNA via RNA interference (RNAi), wherein: a. said siNA molecule is about 19 to about 23 nucleotides in length; and b. said siNA molecule comprises a nucleotide sequence having sufficient complementarily to said TEB4 gene for the siNA molecule to direct cleavage of the TEB4 RNA via RNA interference.

2. The siNA molecule of claim 1 having two strands, wherein one strand of said double-stranded siNA molecule comprises a nucleotide sequence that is complementary to a nucleotide sequence of a TEB4 gene or a portion thereof, and wherein a second strand of said double-stranded siNA molecule comprises a nucleotide sequence complementary to the first strand nucleotide sequence or a portion thereof.

3. The siNA molecule of claim 2 wherein each strand of the siNA molecule comprises about 19 to about 23 nucleotides, and wherein each strand comprises at least about 19 nucleotides that are complementary to the nucleotides of the other strand.

4. An siRNA capable of reducing expression of TEB4 in a cell, comprising the sequence selected from the group consisting of SEQ ID NO: 3-14.

5. The siRNA of claim 4, comprising of SEQ ID NO: 4.

6. The use of the siRNA of claims 1-4 as research reagent.

7. The use of the siRNA of claims 1-4 as diagnostic agent.

8. The use of the siRNA of claims 1-4 in a pharmaceutical composition.

9. A method of preventing, treating or alleviating TEB4 dependent conditions in an individual, which comprises administrating a therapeutically effective amount and in a suitable pharmacological carrier, an siNA compound of claim 1 so that expression of TEB4 is suppressed, thereby suppressing TEB4 dependent processes.

10. The method of claim 9, wherein the TEB4 dependent condition is selected from a proliferative disease, a hypoxia-induced disease, an angiogenesis-related disease, and a metastatic disease.

11. The method of claim 10, wherein the TEB4 dependent condition is cancer.

12. A method of preventing, treating or alleviating TEB4 dependent conditions in an individual, which comprises the extraction of cells, tissue or entire organs from said individual; contacting the said cells, tissue or entire organs with an siNA compound of claim 1, so that expression of the TEB4 is suppressed, thereby suppressing TEB4 dependent processes; and reintroducing the same.

13. The method according to claim 9, for treating a disorder involving epithelial cell proliferation.
